(19) United States
(12) Patent Application Publication
(10) Pub. No.: US 2007/0065390 A1
(43) Pub. Date: Mar. 22, 2007

(54) Stable emulsion systems with high salt tolerance

(76) Inventors: Eric Spengler, Ridgefield, CT (US); Teresa Petraia, Norwalk, CT (US)

Correspondence Address: FITZPATRICK CELLA HARPER & SCINTO, 30 ROCKEFELLER PLAZA, NEW YORK, NY, 10112, US

(21) Appl. No.: 11/370,027

(22) Filed: Mar. 8, 2006

Related U.S. Application Data
(60) Provisional application No. 60/717,751, filed on Sep. 19, 2005.

(51) Int. Cl. A61K 8/49

(52) U.S. Cl. 424/70.21

(57) ABSTRACT

A salt-tolerant, stable emulsion system having low viscosity, as well as method of making the same, is disclosed. The emulsion system may be used for cosmetic and dermatological applications and provides unique benefits.

This application claims the benefit of U.S. Provisional Patent Application No. 60/717,751, filed Sep. 19, 2005.

### BACKGROUND OF THE INVENTION

1. Field of the Invention

This invention relates to stable, low viscosity emulsion systems with high salt tolerance, as well as to a method of making the same and to skin care compositions comprising the same.

2. Related Background Art

Oil-in-water (o/w) emulsions are the most popular type of over-the-counter and personal care emulsions sold in the market today. In such emulsions, the external phase is aqueous, including water and water soluble components, and the internal phase is oil and oil soluble components. The consumer benefit of these formulations is a less greasy skin feel and a light texture relative to oil external phase emulsions. For this reason, systems with high shear thinning (high thixotropy) and low viscosities are very popular. In addition, o/w emulsions are also a desired vehicle for delivering water soluble active ingredients to skin. However, many water soluble active ingredients are salts, and use of ionized compounds at high concentrations present unique formulation and stability challenges.

Emulsions, by design, are thermodynamically unstable. From the point of manufacture, these systems undergo constant change and become progressively unstable. Work to stabilize these emulsions has focused on:

increasing the viscosity of the continuous/external phase

reducing the droplet size via mechanical energy or choice of emulsifiers (i.e., to lower the interfacial tension)

formation of steric barriers with emulsifiers (i.e., mixed emulsifier systems creating a tighter packing at the interface)

creating a controlled charge at the interface and thus forming a repulsive electrical potential (a.k.a. zeta potential).

High concentrations of ionized materials in solution can destabilize the emulsion interface. In fact, adding salt to emulsified materials is a common strategy used by water treatment plants to destabilize emulsions and separate or purify the materials therein.

A common strategy to stabilize emulsions containing high concentrations of ionized materials is to increase the external phase sufficiently to reduce the mobility of the internal phase droplets. However, this strategy does not work to achieve low viscosity formulations.

Accordingly, a stable, low viscosity lotion having excellent salt tolerance is highly desirable. To the inventors' knowledge, no such emulsion system, as set forth in the present invention, exists in the market today.

### SUMMARY OF THE INVENTION

The present invention is directed to a salt-tolerant, stable emulsion system comprising (a) at least one salt present in an amount ranging from about 0.005 to about 2.000 moles/liter, (b) at least one hydrocolloid, and (c) a nanoemulsion. In certain preferred embodiments, the salt is present in an amount ranging from about 0.02 to about 0.5 mole/liter, most preferably from about 0.02 to about 0.1 mole/liter.

In other preferred embodiments the at least one salt is selected from dibucaine HCl, diphenhydramine HCl, lidocaine HCl, tetracaine HCl, pramoxine HCl, dyclonine HCl, dimethisoquin HCl, tripelennamine HCl, benzethonium chloride, arginine HCl, cysteine HCl, histidine HCl, lysine HCl, carnitine HCl, ephedrine HCl, ephedrine sulfate, oxymetazoline HCl, phenylephrine HCl, naphazoline HCl, xylometazoline HCl, phenolate sodium, stearalkonium HCl, ornithine HCl, methyltryptophanate HCl, methyl tyrosinate HCl, 2,4-diaminophenol HCl, glucosamine HCl, guanidine HCl, methenammonium chloride, spermidine HCl, phytosphingosine HCl, cysteine HCl, methyltyrosinate HCl, decarboxy carnosine HCl, pyridoxine HCl, thiamine HCl, benzalkonium chloride, cetrimonium chloride, choline chloride, cocotrimonium chloride, steartrimonium chloride, calcium fluoride, magnesium carbonate, sea salts, mineral salts, sodium bisulfite, zinc chloride and combinations thereof.

In certain preferred embodiments of the invention, the at least one hydrocolloid is a polyacrylate and the at least one hydrocolloid is present in an amount sufficient to achieve a salt-tolerant, stable emulsion system viscosity ranging from about 500 to about 50,000 centipoise, more preferably ranging from about 2,000 to about 30,000 centipoise, and most preferably from about 8,000 to about 20,000 centipoise.

In still other preferred embodiments, the nanoemulsion comprises at least two nonionic emulsifiers, at least one lipophilic ingredient and water.

The present invention is further directed to a skin care composition comprising the salt-tolerant, stable emulsion system of the present invention. In certain preferred embodiments, the skin care composition further comprises ingredients selected from the group consisting of humectants, preservatives, fragrance, color, natural extracts, and combinations thereof.

The present invention is still further directed to a method of making a salt-tolerant, stable emulsion system comprising the steps of (a) providing a nanoemulsion; (b) combining the nanoemulsion with at least one hydrocolloid to form an emulsion system; and (c) dissolving at least one salt present in an amount ranging from about 0.005 to about 2.000 moles/liter in the emulsion system to form a salt-tolerant, stable emulsion system.

The present invention is also directed to salt-tolerant, stable emulsion system made according to the present inventive method.

### DETAILED DESCRIPTION OF THE INVENTION

As used herein, “stable” refers to the absence of significant change in droplet/particle size distribution or the absence of visible phase separation for a period prior to use which is necessary for storage and/or display. Such a degree of stability can be predicted by the absence of visible phase separation for a period of at least 1 week at 50° C., more preferably at 60° C. As used herein, “nanoemulsion” refers to an emulsion typically having a particle size of less than 1 μm, i.e., a sub-micron emulsion, preferably between about 10 nm and about 900 nm, and more preferably between about 50 nm and about 400 nm. As used herein, “HLB” refers to hydrophilic/lipophilic balance (as defined by ICI; see “Uniqema Celebrates 50th Anniversary of HLB System: Company Honors Developer of Valuable Formulation Tool”, Uniqema press release, Wilmington, Del., Jul. 28, 1999; http://www.uniqema.com/press/news8993.htm). As used herein, “semisolid dispersion” refers to the discontinuous phase of an emulsion, i.e., a droplet, a dispersed phase. As used herein, “oil phase” refers to the combination of emulsifier(s) and lipophilic material(s) used to form semisolid dispersions.

The first embodiment of the present invention is directed to a salt-tolerant, stable emulsion system comprising (a) at least one salt present in an amount ranging from about 0.005 to about 2.000 moles/liter, (b) at least one hydrocolloid and (c) a nanoemulsion. The present invention specifically relates to a novel emulsion system with high salt tolerance; in other words, a salt content ranging from about 0.005 to about 2.000 moles/liter, more preferably from about 0.02 to about 0.5 mole/liter, most preferably from about 0.02 to about 0.1 mole/liter, does not destabilize the emulsion of the present invention.

As used herein, “salt” refers to any material which is formed when the hydrogen of an acid is replaced by a metal or its equivalent and which becomes ionized when dissolved in water at the appropriate pKa. While salts suitable for use in this invention are broadly directed to any type of salt of any type of material suitable for use in an emulsion system or skin care composition, the key salts of interest are drugs or active components which provide a physiologic action on skin or mucous membranes. Interestingly, virtually all drug-like molecules are weak acids or bases. As used herein, “weak acids or bases” refers to molecules which contain at least one site that can reversibly disassociate or associate a proton (a hydrogen ion) to form a negatively charged anion or a positively charged cation; molecules that disassociate protons are acids, and those that associate protons are bases. The reversibility means that a sample is always in an equilibrium with some fraction protonated and the rest deprotonated:
HA<=>H++A− or HB+<=>H++B

By varying the availability of protons, i.e. the acidity (or pH adjustment) of a given media, the balance of the equilibrium can be shifted. This provides a measure of the ease of proton disassociation of a site in a compound, the disassociation (or ionization) constant pKa, defined by the equation:
pKa=pH+log(protonated/unprotonated)

Alternatively, the pKa of a site can be thought of the pH at which the protonated and deprotonated fractions are equal. If the pH is higher than the pKa, the site is mostly deprotonated, and if the pH is lower than the pKa, the site is mostly protonated. See, R. Sayle, “Physiological Ionization and pKa Prediction”, Metaphorics LLC, Bioinformatics Group, Santa Fe, N. Mex.; http://www.daylight.com/meetings/emug00/Sayle/pkapredict.html.

Particularly suitable for use in the present invention are salts of weak bases. Preferably the salt is selected from skin care and oral care agents which provide a physiologic action on skin or mucous membranes such as anesthetics, bronchodilators, humectants and the like. Typical salts include, without limitation, dibucaine HCl, diphenhydramine HCl, lidocaine HCl, tetracaine HCl, pramoxine HCl, dyclonine HCl, dimethisoquin HCl, tripelennamine HCl, benzethonium chloride, arginine HCl, cysteine HCl, histidine HCl, lysine HCl, carnitine HCl, ephedrine HCl, ephedrine sulfate, oxymetazoline HCl, phenylephrine HCl, naphazoline HCl, xylometazoline HCl, phenolate sodium, stearalkonium HCl, ornithine HCl, methyltryptophanate HCl, methyl tyrosinate HCl, 2,4-diaminophenol HCl, glucosamine HCl, guanidine HCl, methenammonium chloride, spermidine HCl, phytosphingosine HCl, cysteine HCl, methyltyrosinate HCl, decarboxy carnosine HCl, pyridoxine HCl, thiamine HCl, benzalkonium chloride, cetrimonium chloride, choline chloride, cocotrimonium chloride, steartrimonium chloride, calcium fluoride, magnesium carbonate, sea salts, mineral salts, sodium bisulfite, zinc chloride and combinations thereof.

Diphenhydramine HCl and dibucaine HCl, for example, have a high pKa (˜9); the pH of a given system is thus much lower at the physiologic pH of skin (pH 4.5-5.5) or at the pH of most emulsion systems (pH 4-8), making the active protonated. Without being bound to any particular theory, we believe it is the relatively high concentration of protonated molecules which makes stabilizing a salt-containing emulsion system a challenge and the success of the present invention remarkable.

The hydrocolloid component of the inventive emulsion system is preferably employed in an amount sufficient to result in a salt-tolerant, stable emulsion system viscosity ranging from about 500 to about 50,000 centipoise, more preferably in an amount sufficient to result in a viscosity ranging from about 2,000 to about 30,000 centipoise, and most preferably in an amount sufficient to result in a viscosity ranging from about 8,000 to about 20,000 centipoise. Viscosity can be measured using any suitable measurement technique/apparatus such as by using a Brookfield RVT Viscometer (Brookfield Engineering Laboratories, Inc., Middleboro, Mass.) with a #5 spindle at 10 rpm.

The hydrocolloid used in the present invention is preferably a polyacrylate. U.S. Patent Application Publication No. 2004/0202635, the entire disclosure of which is incorporated by reference herein, contains an effective description of such hydrocolloids suitable for use in this invention. In particular, acrylate copolymers and/or acrylate-alkyl acrylate copolymers which are available under the marks Carbopol® 1382, Carbopol® 981, Carbopol® 5984, Aqua™ SF-1 (NOVEON Inc.), and Aculyn® 33 (Rohm & Haas). Also useful as the hydrocolloid of the present invention are copolymers of C₁₀₋₃₀-alkyl acrylates and one or more monomers of acrylic acid, of methacrylic acid or esters thereof which are crosslinked with an alkyl ether of sucrose or an alkyl ether of pentaerythritol. Additionally, compounds which carry the INCI name “acrylates/C₁₀₋₃₀ alkyl acrylate crosspolymer” are advantageous. Particularly advantageous are those polymers available under the marks Pemulen™ TR1 and Pemulen™ TR2 from NOVEON Inc., Ultrez™ 21 and Carbopol® ETD 2020. What is more, compounds which carry the INCI name “acrylates/C₁₂₋₂₄ pareth-25 acrylate copolymer” (obtainable under the mark Synthalen® W2000 from 3V Inc.), the INCI name “acrylates/steareth-20 methacrylate copolymer” (obtainable under the mark Aculyn® 22 from Rohm & Haas), the INCI name “acrylates/steareth-20 itaconate copolymer” (obtainable under the mark Structure 2001® from National Starch), the INCI name “acrylates/aminoacrylates/C₁₀₋₃₀ alkyl PEG-20 itaconate copolymer” (obtainable under the mark Structure Plus® from National Starch) and similar polymers are also useful for purposes of the present invention. The hydrocolloids preferred for use in the present invention include Carbopol® ETD2020 and Ultrez™ 21.

Nanoemulsions suitable for use in the present invention include typical or conventional ingredients and can be made by any known method, i.e., phase inversion (PIT), high pressure homogenization, low energy emulsification, and the like. Accordingly, any nanoemulsion can be incorporated into the salt-tolerant, stable emulsion system of the present invention so long as the nanoemulsion is itself stable prior to its incorporation into the present inventive system.

In a preferred embodiment of the invention, the nanoemulsion comprises at least two nonionic emulsifiers, at least one lipophilic ingredient and water. Preferably the weight ratio of lipophilic ingredient to emulsifier in the nanoemulsion semisolid dispersions ranges from about 1:1 to about 20:1, preferably from about 2:1 to about 10:1, and more preferably from about 3:1 to about 6:1. Preferably, at least one emulsifier has a high HLB of 8 or more, preferably ranging from about 14 to about 15, and the other emulsifier has a low HLB of below 8, preferably about 4.

Nonionic emulsifiers suitable for use in the nanoemulsion component of the present invention are quite diverse; preferably, they are limited only by their ability to satisfy the above-noted HLB parameters. Suitable nonionic emulsifiers or surfactants can be found in Surfactants in Cosmetics, 2d edition, M. Rieger et al., eds., Marcel Dekker, Inc., New York, pp. 19-28 (1997) and in Harry's Cosmetology, 8^{th} edition, M. Rieger, ed., Chemical Publishing Co., Inc., New York, pp. 202-209 (1997), the pertinent disclosure of each of which is incorporated by reference herein. Generally, nonionic surfactants are substances in which the molecule carries no charge. The hydrophobe can be highly variable, but the hydrophilic head generally includes a polyether group or at least one —OH group.

The nonionic surfactants most useful for purposes of the present invention can be conveniently divided into three large groups. The first of these groups is alcohols [R—CH₂—OH], for example, cetearyl alcohol; preferably the alkyl R group has a chain length ranging from 6-22 carbons.

The second of these groups is esters. Esters include glycerides such as glyceryl stearate and glyceryl oleate; ethoxylated glycerides such as PEG-20 glyceryl stearate; polyglyceryl esters such as polyglyceryl-2-caprate; sorbitan esters such as Tween 80 and sorbitan oleate; carbohydrate esters such as sucrose distearate and PEG-120 methyl glucose dioleate; ethoxylated carboxylic acids such as ethoxylated fatty acids like PEG-150 oleate and PEG-6 dilaurate; and phosphoric acid triesters such as trideceth-3 phosphate. Further, a large number of nonionic esters can be prepared by reaction of alcohols or polyalcohols with a variety of natural and or hydrogenated oils, i.e., via alcohol-oil transesterification. Most commonly, the oils used are castor oil or hydrogenated castor oil, or an edible vegetable oil such as corn oil. Preferred alcohols include glyceryol, propylene glycol, ethylene glycol, polyethylene glycol, sorbitol and pentaerythritol. Examples of transesterified nonionic surfactants include, without limitation, PEG-40 hydrogenated castor oil, PEG-60 corn glycerides, and PEG-40 palm kernel oil.

The third of these groups is ethers. Ethers include ethoxylated alcohols such as laureth 4, ceteareth-10 and ceteareth-20; ethoxylated (propoxylated) polysiloxanes such as dimethicone copolyols and PEG/PPG-15/15 dimethicone; ethoxylated polypropylene oxide ethers such as poloxamer 407, PPG-9 buteth-12; and alkyl glycosides such as decyl glucoside.

Exact carbon chain lengths and the degree of propoxylation/ethoxylation for all of the above-noted surfactants will determine the HLB value of nonionic emulsifiers such as these. Ultimately it is important to note that a broad spectrum of nonionic emulsifiers can be used for purposes of this invention and that such emulsifiers preferably satisfy the HLB parameters set forth above.

Lipophilic ingredients suitable for use in the nanoemulsion component of the invention include, without limitation, aliphatic hydrocarbons (straight or branched chain) such as mineral oil and isododecane; natural oils such as soybean oil, sunflower seed oil, olive oil, palm oil, wheat germ oil, shark liver oil, squalene, shea butter; esters such as isopropyl myristate; branched chained esters, e.g., chain length from 3-30, such as cetyl ethylhexanoate; waxes such as beeswax, jojoba wax, and carnuba wax; silicones; active ingredients; and

mixtures thereof.

Lipophilic active ingredients as noted above may include actives such as anti-inflammatory agents, both steroidal (hydrocortisone, beclomethasone, etc.) and non-steroidal (oxicams, salicylates, acetic acid derivatives, fenamates, propionic acid derivatives, pyrazoles) as well as natural anti-inflammatory agents (aloe vera, bisabolol, glycyrrhetinic acid, etc.); antioxidants (ursoic acid, tocopherol, etc.); oil soluble vitamins (D, A, folic acid, etc.); topical anesthetics (benzocaine, lidocaine, etc.); antimicrobial agents (phenolic cosmetic biocide); antifungal agents; sunscreen agents (physical blockers such as metallic oxides like titanium and zinc oxides; and UVA & UVB absorbers such as octyl methoxycinnamate, avobenzone, 4-methylbenzylidene camphor); skin-lightening agents; anti-acne agents (salicylic acid, benzoyl peroxide, azelaic acid, isotretinoin, etc.); antibiotics (clindamycin, erythromycin, metronidazol, sulfacetamide, etc.) and combinations thereof. U.S. Pat. No. 6,492,326, the disclosure of which is incorporated by reference herein, sets forth a useful discussion with regard to lipophilic active ingredients which may advantageously be employed for use in the present invention.

Also present in the nanoemulsion component of the first embodiment of the invention is water. Water is preferably employed in an amount ranging from 40% to 99%, more preferably from about 75% to about 95%, and most preferably from about 80% to about 90%, by weight of the stable emulsion system.

Other ingredients suitable for use in the salt-tolerant, stable emulsion system of the present invention include, without limitation, humectants, hydrocolloid/rheology modifiers, actives, color, fragrance, preservatives, antioxidants, chelators, aqueous actives, anionic hydrocolloid neutralizers (such as triethanolamine and sodium hydroxide), water soluble natural extracts, water soluble active ingredients, water soluble vitamins, and combinations thereof. One of ordinary skill in the art would readily be able to determine the amount of these other ingredients suitable for use in the salt-tolerant, stable emulsion system of the present invention based on the desired end product.

The emulsion systems of the present invention are stable. In other words, there is no physical separation for a period prior to use which is necessary for storage and/or display, where such a degree of stability can be predicted by the absence of significant change in droplet/particle size distribution or the absence of visible phase separation as determined in accordance with International Conference on Harmonization (ICH) guidelines or for a period of at least 1 week at 50° C., more preferably at 60° C. Even more preferably, the emulsion systems of the present invention are stable over a temperature range from about −15° C. to about 60° C. In addition to stability, the emulsion systems of the present invention exhibit excellent organoleptic properties, i.e., skin feel, and satisfy the desire to deliver salt-based drugs in an elegant, low-viscosity lotion form. The low-viscosity of the emulsion systems of the present invention allow for the formulation of light, quick-absorbing, non-greasy lotions.

A second embodiment of the present invention is directed to a skin care composition comprising a salt-tolerant, stable emulsion system in accordance with the first embodiment of this invention. For purposes of this invention, “skin care composition” refers to a topical composition which can be applied to any or all of skin, mucous membranes, hair, etc., for any purpose, i.e., a lotion. The skin care composition may additionally comprise other typical ingredients. Other typical ingredients include, without limitation, humectants, preservatives, fragrance, color, natural extracts, and combinations thereof. The salt-tolerant, stable emulsion system of the first embodiment of the invention is typically employed in an amount ranging from 99.99% to 1.0% by weight of a skin care composition of the second embodiment.

The third embodiment of the present invention is directed to a method of making a salt-tolerant, stable emulsion system. More specifically, the method comprises (a) providing a nanoemulsion, (b) combining the nanoemulsion with at least one hydrocolloid to form an emulsion system, and (c) dissolving at least one salt present in an amount ranging from about 0.005 to about 2.000 moles/liter in the emulsion system to form a salt-tolerant, stable emulsion system. The nanoemulsion of step (a) is preferably made using a PIT method, though a nanoemulsion made by any other process can be used for purposes of the present invention. Typically the hydrocolloid in step (b) is well-hydrated prior to its combination with the nanoemulsion.

The fourth embodiment of the present invention is directed to a salt-tolerant, stable emulsion system made according to the inventive method of the third embodiment.

All of the details regarding the second, third and fourth embodiments of the present invention, i.e., amount of salt, type and amount of hydrocolloid, types of emulsifiers, etc., are the same as those regarding the first embodiment of the invention set forth above.

Specific embodiments of the invention will now be demonstrated by reference to the following examples. It should be understood that these examples are disclosed solely by way of illustrating the invention and should not be taken in any way to limit the scope of the present invention.

### EXAMPLE 1

A salt-tolerant, stable emulsion system was made to contain the components as set forth in Table 1 below.

TABLE 1

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| isopropyl methyl phenol | 0.10 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| carbomer | 0.80 |
| (Carbopol ETD2020) | |
| preservatives, chelator | q.s. |
| D. miscellaneous | |
| neutralizer, color, fragrance | q.s. |
| E. actives | |
| diphenhydramine HCl | 1.00 |
| dibucaine HCl | 1.00 |

Phase A ingredients were combined in a suitably sized vessel equipped with a mixer and heating capability. The ingredients were heated to 90-100° C. while mixing with high shear. Phase B water was heated to 90-110° C. with mixing. Then Phase A was added to Phase B with high shear mixing. Next, the mixture was cooled to 30-40° C. while mixing slowly to form a PIT nanoemulsion.

Phase C water was put in a suitably sized vessel and, with moderate mixing, the carbomer was added to the water. Mixing continued until the hydrocolloid was hydrated. Then, the remaining Phase C ingredients were added and mixed until homogenous.

Next the PIT nanoemulsion was added to Phase C with mixing. Finally, Phase D and E ingredients were added, mixing after each addition until homogenous.

### EXAMPLE 2

A salt-tolerant, stable emulsion system was made to contain the components as set forth in Table 2 below using the method of Example 1.

TABLE 2

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| acrylates/C₁₀₋₃₀ alkyl acrylate | 0.80 |
| crosspolymer | |
| preservatives, chelator | q.s. |
| D. miscellaneous | |
| neutralizer, color, fragrance | q.s. |
| E. active | |
| diphenhydramine HCl | 2.00 |

### EXAMPLE 3

A salt-tolerant, stable emulsion system was made to contain the components as set forth in Table 3 below using the method of Example 1.

TABLE 3

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| acrylates/C₁₀₋₃₀ alkyl acrylate | 0.80 |
| crosspolymer | |
| preservatives, chelator | q.s. |
| D. miscellaneous | |
| neutralizer, color, fragrance | q.s. |
| E. active | |
| dibucaine HCl | 1.00 |

### EXAMPLE 4

A salt-tolerant, stable emulsion system was made to contain the components as set forth in Table 4 below using the method of Example 1.

TABLE 4

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| acrylates/C₁₀₋₃₀ alkyl acrylate | 0.80 |
| crosspolymer | |
| preservatives, chelator | q.s. |
| D. miscellaneous | |
| neutralizer, color, fragrance | q.s. |
| E. active | |
| lidocaine HCl | 4.00 |
| benzethonium chloride | 0.20 |

### EXAMPLE 5

A salt-tolerant, stable emulsion system was made to contain the components as set forth in Table 5 below using the method of Example 1.

TABLE 5

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| shea butter | 0.50 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| acrylates/C₁₀₋₃₀ alkyl acrylate | 0.80 |
| crosspolymer | |
| preservatives, chelator | q.s. |
| D. miscellaneous | |
| neutralizer, color, fragrance | q.s. |
| E. active | |
| arginine HCl | 0.50 |
| cysteine HCl | 0.50 |
| lysine HCl | 0.50 |
| L-ornithine | 0.40 |
| pyridoxine HCl | 0.20 |

### EXAMPLE 6

A salt-tolerant, stable emulsion system was made to contain the components as set forth in Table 6 below using the method of Example 1.

TABLE 6

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| acrylates/steareth-20 methacrylate | 1.00 |
| copolymer | |
| preservatives, chelator | q.s. |
| D. miscellaneous | |
| neutralizer, color, fragrance | q.s. |
| E. active | |
| dibucaine HCl | 1.00 |

### COMPARATIVE EXAMPLE 1

A nanoemulsion was made to contain the components as set forth in Table 7 below using the method of Example 1 except that hydroxyethylcellulose is employed in Phase C herein.

TABLE 7

| Ingredients | % w/w |
|---|---|
| A. PIT oil phase | |
| PEG-40 hydrogenated castor oil | 0.90 |
| ceteareth-20 | 0.30 |
| glyceryl oleate | 0.30 |
| cetyl ethylhexanoate | 3.80 |
| isopropyl methyl phenol | 0.10 |
| glycyrrhetinic acid | 0.10 |
| vitamin E acetate | 0.50 |
| B. PIT water phase | |
| deionized water | 20.0 |
| C. hydrocolloid phase | |
| deionized water | q.s.a.d. |
| glycerin | 3.00 |
| hydroxyethylcellulose | 0.50 |
| (Natrosol 250HX) | |
| disodium EDTA | 0.20 |
| preservatives | q.s. |
| D. miscellaneous | |
| diphenhydramine HCl | 1.00 |
| dibucaine HCl | 1.00 |

### COMPARATIVE EXAMPLE 2

A macroemulsion was made to contain the components as set forth in Table 8 below.

TABLE 8

| Ingredients | % w/w |
|---|---|
| A. oil phase | |
| cetyl alcohol | 0.50 |
| steareth-21 | 3.00 |
| steareth-2 | 1.00 |
| caprylic/capric triglyceride | 5.00 |
| fumed silica | 0.25 |
| dimethicone | 1.00 |
| isopropyl methyl phenol | 0.10 |
| glycyrrhetinic acid | 0.10 |
| vitamin E acetate | 0.50 |
| B. water phase | |
| deionized water | q.s.a.d. |
| glycerin | 3.00 |
| hydroxyethylcellulose | 0.50 |
| (Natrosol 250HX) | |
| preservatives | q.s. |
| C. miscellaneous | |
| diphenhydramine HCl | 1.00 |
| dibucaine HCl | 1.00 |

Phase A ingredients were combined in a suitably sized vessel equipped with a mixer and heating capability. The ingredients were heated to 85-90° C. while mixing. Phase B water was heated to 75-80° C. with mixing. The preservatives were added to the water and mixed until dissolved. The hydrocolloid was added to the water and mixed until hydrated. Then, the glycerin was added and mixed until homogeneous. When both Phase A and Phase B were at temperature and homogeneous, Phase A was added to Phase B with mixing. Then the mixture was mixed for 15 minutes at 75-80° C. Next, the mixture was cooled to 30-40° C. while mixing slowly. The remaining miscellaneous ingredients were added, mixing well after each addition until homogeneous.

### COMPARATIVE EXAMPLE 3

A macroemulsion was made to contain the components as set forth in Table 9 below according to the process of Comparative Example 2, with the note that the neutralizer was added last.

TABLE 9

| Ingredients | % w/w |
|---|---|
| A. oil phase | |
| cetyl alcohol | 0.50 |
| steareth-21 | 3.00 |
| steareth-2 | 1.00 |
| caprylic/capric triglyceride | 5.00 |
| fumed silica | 0.25 |
| dimethicone | 1.00 |
| isopropyl methyl phenol | 0.10 |
| glycyrrhetinic acid | 0.10 |
| vitamin E acetate | 0.50 |
| B. water phase | |
| deionized water | q.s.a.d. |
| glycerin | 3.00 |
| carbomer | 0.80 |
| (Carbopol ETD2020) | |
| preservatives | q.s. |
| C. miscellaneous | |
| neutralizer | q.s. |
| diphenhydramine HCl | 1.00 |
| dibucaine HCl | 1.00 |

### Stability Testing

The nanoemulsion of Example 1 and the emulsions of Comparative Examples 1-3 were tested for stability according to ICH guidelines. In addition, each sample was tested for a period of one week at 50° C. and 60° C. The salt-tolerant, stable emulsion system of Example 1 exhibited no physical separation of water and oil, while each of Comparative Examples 1-3 was unstable (i.e., physical separation of water and oil was exhibited).

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A salt-tolerant, stable emulsion system comprising:
(a) at least one salt present in an amount ranging from about 0.005 to about 2.000 moles/liter;
(b) at least one hydrocolloid; and
(c) a nanoemulsion.

2. The salt-tolerant, stable emulsion system of claim 1, wherein the salt is present in an amount ranging from about 0.02 to about 0.5 mole/liter.

3. The salt-tolerant, stable emulsion system of claim 2, wherein the salt is present in an amount ranging from about 0.02 to about 0.1 mole/liter.

4. The salt-tolerant, stable emulsion system of claim 1, wherein the at least one salt is selected from the group consisting of dibucaine HCl, diphenhydramine HCl, lidocaine HCl, tetracaine HCl, pramoxine HCl, dyclonine HCl, dimethisoquin HCl, tripelennamine HCl, benzethonium chloride, arginine HCl, cysteine HCl, histidine HCl, lysine HCl, carnitine HCl, ephedrine HCl, ephedrine sulfate, oxymetazoline HCl, phenylephrine HCl, naphazoline HCl, xylometazoline HCl, phenolate sodium, stearalkonium HCl, ornithine HCl, methyltryptophanate HCl, methyl tyrosinate HCl, 2,4-diaminophenol HCl, glucosamine HCl, guanidine HCl, methenammonium chloride, spermidine HCl, phytosphingosine HCl, cysteine HCl, methyltyrosinate HCl, decarboxy carnosine HCl, pyridoxine HCl, thiamine HCl, benzalkonium chloride, cetrimonium chloride, choline chloride, cocotrimonium chloride, steartrimonium chloride, calcium fluoride, magnesium carbonate, sea salts, mineral salts, sodium bisulfite, zinc chloride and combinations thereof.

5. The salt-tolerant, stable emulsion system of claim 1, wherein the at least one hydrocolloid is a polyacrylate.

6. The salt-tolerant, stable emulsion system of claim 1, wherein the at least one hydrocolloid is present in an amount sufficient to achieve a salt-tolerant, stable emulsion system viscosity ranging from about 500 to about 50,000 centipoise.

7. The salt-tolerant, stable emulsion system of claim 6, wherein the viscosity ranges from about 2,000 to about 30,000 centipoise.

8. The salt-tolerant, stable emulsion system of claim 7, wherein the viscosity ranges from about 8,000 to about 20,000 centipoise.

9. The salt-tolerant, stable emulsion system of claim 1, wherein the nanoemulsion comprises at least two nonionic emulsifiers, at least one lipophilic ingredient and water.

10. The salt-tolerant, stable emulsion system of claim 1, wherein the salt-tolerant, stable emulsion system is stable over period of one week and over a temperature range from −15° C. to 60° C.

11. A skin care composition comprising the salt-tolerant, stable emulsion system of claim 1.

12. The skin care composition of claim 11 further comprising ingredients selected from the group consisting of humectants, preservatives, fragrance, color, natural extracts, and combinations thereof.

13. A method of making a salt-tolerant, stable emulsion system comprising the steps of:
(a) providing a nanoemulsion;
(b) combining the nanoemulsion with at least one hydrocolloid to form an emulsion system; and
(c) dissolving at least one salt present in an amount ranging from about 0.005 to about 2.000 moles/liter in the emulsion system to form a salt-tolerant, stable emulsion system.

14. A salt-tolerant, stable emulsion system made according to the method of claim 13.
